(19) United States
(12) Patent Application Publication
(10) Pub. No.: US 2011/0250122 A1
(43) Pub. Date: Oct. 13, 2011

(54) Core-Shell Nanocatalyst For High Temperature Reactions

(75) Inventors: Sang Hoon Joo, Ulsan (KR); Jeong Young Park, Albany, CA (US); Chia-Kuang Tsung, Aliston, MA (US); Peidong Yang, Kensington, CA (US); Gabor A. Somorjai, Berkeley, CA (US)

(73) Assignee: The Regents of the University of California, Oakland, CA (US)

(21) Appl. No.: 13/128,226

(22) PCT Filed: Nov. 3, 2009
(86) PCT No.: PCT/US09/63160

§ 371 Date: Jun. 29, 2011

Related U.S. Application Data
(60) Provisional application No. 61/112,607, filed on Nov. 7, 2008.

(51) Int. Cl. C01B 31/20; B01J 23/42; B01J 23/44; B01J 23/46; B01J 23/52; B01J 23/50; B01J 23/72; B01J 23/755; B01J 23/75; B01J 23/745; B01J 21/08; B01J 21/04; B01J 21/06; B01J 23/20; B01J 35/08; B01J 35/06; B01J 37/025; B01J 37/08; B01J 37/14; C07C 5/03; B01J 35/10; B82Y 30/00

(52) U.S. Cl. 423/437.2; 502/300; 502/339; 502/325; 502/344; 502/347; 502/345; 502/337; 502/338; 502/240; 502/355; 502/350; 502/349; 502/353; 585/275; 977/773; 977/774; 977/762

(57) ABSTRACT

The present invention provides a core-shell nanoparticle that includes a metal-oxide shell and a nanoparticle. Pores extend from an outer surface to an inner surface of the shell. The inner surface of the shell forms a void, which is filled by the nanoparticle. The pores allow gas to transfer from outside the shell to a surface of the nanoparticle. The present invention also provides a method of making a core-shell nanoparticle includes forming a metal-oxide shell on a colloidal nanoparticle, which forms a precursor core-shell nanoparticle. A capping agent is removed from the precursor core-shell nanoparticle, which produces the core-shell nanoparticle. The present invention also provides a method of using a nanocatalyst of the present invention includes providing the nanocatalyst, which is the core-shell nanoparticle. Reactants are introduced in a vicinity of the nanocatalyst, which produces a reaction that is facilitated or enhanced by the nanocatalyst.

### RELATED APPLICATIONS

This application claims the benefit of and priority to U.S. Provisional Application No. 61/112,607, filed on Nov. 7, 2008, which is hereby incorporated by reference.

### STATEMENT OF GOVERNMENT SUPPORT

This invention was made with government support under Contract No. DE-AC02-05CH11231 awarded by the U.S. Department of Energy. The government has certain rights in this invention.

### BACKGROUND OF THE INVENTION

The present invention relates to the field of nanotechnology and, more particularly, to the field of nanotechnology that includes core-shell particles and catalysis.

Recent advances in colloidal synthesis has enabled the precise control of size, shape and composition of catalytic metal nanoparticles, allowing their use as model catalysts for systematic investigations of the atomic-scale properties affecting catalytic activity and selectivity. The organic capping agents that stabilize colloidal nanoparticles, however, often limit their application in high-temperature catalytic reactions.

To design high performance catalysts in terms of activity, selectivity and resistance to deactivation, one should understand the properties that affect catalytic performance (refs. 1-4). Over the past few decades, model catalytic systems, including metal single crystals and lithographically fabricated metal nanostructures, have successfully been used to uncover atomic-scale characteristics, such as surface structures and particle size, that are critical to catalytic activity and selectivity (refs. 5-8). Recent advances in colloid chemistry allow catalytic nanoparticles to be readily prepared with tunable particle size, shape and composition (refs. 9-13). Starting with colloidal nanoparticles, 2-dimensional (2D) and 3-dimensional (3D) model catalysts have been developed which are composed of arrays of nanoparticles on a flat substrate and nanoparticles dispersed on high-surface-area mesoporous oxide support, respectively. These model catalytic systems have enabled systematic investigations of the effects of particle size (ref. 14), shape (refs. 15-17) and composition (refs. 18, 19) on catalytic properties.

Catalytic studies of colloidal nanoparticles have shown that the thermal and chemical stabilities of nanoparticle catalysts are crucial. Colloidal nanoparticles are usually prepared in the presence of organic capping agents, such as polymers or surfactants, which prevent aggregation of nanoparticles in solution. At high temperatures, typically above 300° C., however, the organic capping layers can decompose and the nanoparticles can deform and aggregate. As a result, the size, shape and composition of nanoparticles during or after high temperature reactions could be different from those of pristine nanoparticles. Many industrially important catalytic processes, including CO oxidation (refs. 20-25), partial oxidation (ref. 26) and cracking (ref 27) of hydrocarbons, and combustion (ref 28) reactions, are performed at temperatures above 300° C. In this regard, model catalysts that are stable at high reaction temperatures are high in demand.

In previous examples of metal-mesoporous silica core-shell particles (refs. 33-35), an intermediate protecting amorphous silica layer was often sandwiched between the metal core and the mesoporous silica layer, thus hampering direct access of reactants to the metal core. Further, core-shell nanoparticles having a noble-metal core and a metal-oxide shell have recently been exploited for catalytic applications (refs. 38-43). Examples include Pt—CoO yolk-shell nanoparticles (ref. 38), PVP capped Pt encapsulated in mesoporous silica (ref 39), Pt nanoparticles entrapped in hollow carbon shells (ref. 40), and Au nanoparticles within hollow zirconia (ref 41) and hollow silica (ref. 42) and tin oxide shells (ref. 43). But these core-shell nanoparticles are believed to have limited catalytic performance due to physical isolation of the core from reactants or to not perform well at high temperature (e.g., greater than 300° C.) due to instability of the core within a hollow shell.

### SUMMARY OF THE INVENTION

Embodiments of the present invention include a core-shell nanoparticle, a method of making a core-shell nanoparticle, and a method of using a core-shell nanoparticle as a nanocatalyst.

An embodiment of a core-shell nanoparticle of the present invention includes a metal-oxide shell and a nanoparticle. The metal-oxide shell includes an outer surface, an inner surface, and pores. The pores extend from the outer surface to the inner surface of the shell. The inner surface of the shell forms a void within the shell. The nanoparticle fills the void within the shell. The pores allow gas to transfer from outside the metal-oxide shell to a surface of the nanoparticle.

An embodiment of a method of making a core-shell nanoparticle of the present invention includes forming a metal-oxide shell on a colloidal nanoparticle. The colloidal nanoparticle includes a nanoparticle and a capping agent on the surface of the nanoparticle. Forming the metal-oxide shell on the colloidal nanoparticle produces a precursor core-shell nanoparticle. The capping agent is removed from the precursor core-shell nanoparticle, which produces the core-shell nanoparticle of the present invention.

An embodiment of a method of using a nanocatalyst of the present invention includes providing the nanocatalyst that is a core-shell nanoparticle of the present invention. Reactants are introduced in a vicinity of the nanocatalyst, which produces a reaction that is facilitated or enhanced by the nanocatalyst.

### BRIEF DESCRIPTION OF THE DRAWINGS

The present invention is described with respect to particular exemplary embodiments thereof and reference is accordingly made to the drawings in which:

FIG. 1. Schematic representation for the synthesis of Pt-mesoporous silica core-shell (Pt@mSiO₂) nanoparticles. Pt nanoparticles were synthesized using TTAB surfactant as the capping agent, and used as the core particles. Secondly, as-synthesized Pt@SiO₂ particles were prepared by polymerizing TEOS around the TTAB-capped Pt cores. The as-synthesized Pt@SiO₂ particles were subsequently converted to Pt@mSiO₂ particles by calcination.

FIG. 2. TEM and XRD characterizations of TTAB-capped Pt and as-synthesized Pt@SiO₂ core-shell nanoparticles. FIG. 2a-2c, TEM images of TTAB-capped Pt (FIG. 2a) and as-synthesized Pt@SiO₂ nanoparticles (FIGS. 2b and 2c). FIG. 2d, high angle XRD patterns of Pt and Pt@SiO₂ nanoparticles.

FIG. 3. Thermal stability of Pt@mSiO₂ nanoparticles. TEM images of Pt@mSiO₂ nanoparticles after calcination at 350° C. (FIGS. 3a and 3b), at 550° C. (FIG. 3c), and at 750° C. (FIG. 3d).

FIG. 4. Structural characterization of Pt@mSiO₂ nanoparticles calcined at 350° C. FIG. 4a, nitrogen adsorption-desorption isotherms. FIG. 4b, pore size distribution calculated from adsorption branch of isotherms.

FIG. 5. CO oxidation activity of TTAB-capped Pt (red hollow square) and Pt@mSiO₂ (blue solid diamond) nanoparticles.

FIG. 6. Change of Pt nanoparticle morphologies before (FIG. 6a-6c) and after (FIG. 6d-6f) CO oxidation at 300° C. SEM images of core-shell Pt@mSiO₂ (FIGS. 6a and 6d) and TTAB-capped Pt (FIGS. 6b and 6e) nanoparticle arrays on 2D silicon wafer, and TEM images of TTAB-capped Pt nanoparticles dispersed on 3D MCF mesoporous silica support (FIGS. 6c and 60.

FIG. 7 illustrates an embodiment of a core-shell nanoparticle of the present invention.

FIG. 8. Pt@SiO₂ core-shell nanoparticles synthesized under (FIG. 8a) lower and (FIG. 8b) higher concentrations of TEOS, compared to the Pt@SiO₂ nanoparticles synthesized under the optimum TEOS concentration where each Pt particle is encaged within a silica shell.

FIG. 9. TEM images of (FIG. 9a) 8.5 nm Pt and (FIG. 9b) corresponding Pt@SiO₂ core-shell nanoparticles. The shell thickness of these nanoparticles was found to be ˜18 nm, similar to that of core-shell nanoparticles with 14 nm Pt core.

FIG. 10. High resolution TEM image Pt core in Pt@mSiO₂ core-shell nanoparticle after calcination at 350° C.

FIG. 11. High resolution TEM image Pt core in Pt@mSiO₂ core-shell nanoparticle after calcination and subsequent CO oxidation.

FIG. 12. XPS plots of Pt@SiO₂ core-shell nanoparticles (FIG. 12a) before and (FIG. 12b) after calcination and CO oxidation. The Pt@SiO₂ nanoparticles were deposited on a silicon wafer using the Langmuir-Blodgett (LB) technique. The maximum temperature for CO oxidation was 340° C. The binding energy for XPS measurements was calibrated with the C1s peak (285 eV). O1s is presumably attributed to the SiO₂ oxygen. The as-synthesized sample exhibits two peaks of hydroxides and atomic oxygen. After calcination, the hydroxide peak decreased and a small peak that is attributed to Pt—O, appeared, suggesting that Pt core is partially oxidized during the catalytic reactions.

FIG. 13 provides a TEM image of an example of a core-shell nanoparticle of the present invention in which the core is a Co nanoparticle and the shell is a mesoporous SiO2 shell.

FIG. 14 provides a TEM image of example core-shell nanoparticles of the present invention in which the core is a Co nanoparticle and the shell is a mesoporous SiO2 shell.

### DETAILED DESCRIPTION OF THE INVENTION

Embodiments of the present invention include a core-shell nanoparticle, a method of making a core-shell nanoparticle, and a method of using a core-shell nanoparticle as a nanocatalyst.

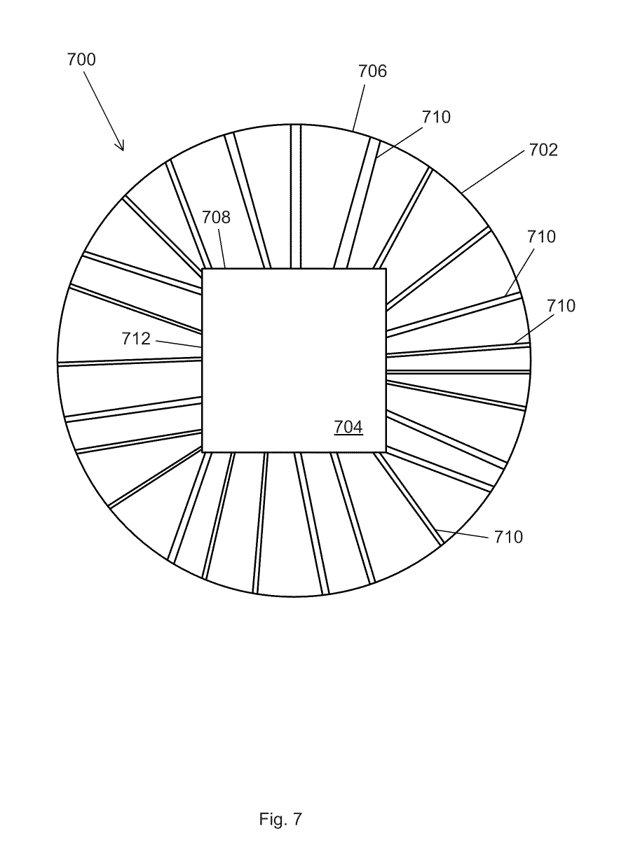

An embodiment of a core-shell nanoparticle of the present invention is illustrated in FIG. 7, as a cross-sectional view. The core-shell nanoparticle 700 includes a metal-oxide shell 702 and a nanoparticle 704. The metal-oxide shell 702 includes an outer surface 706, an inner surface 708, and pores 710. The pores 710 extend from the outer surface 706 to the inner surface 708. The inner surface 708 forms a void within the metal-oxide shell 702. The nanoparticle 704 fills the void within the metal-oxide shell 702. As used herein, the term “nanoparticle” means a particle having a dimension on the nanometer scale. Further, the term “nanoparticle” includes a quantum dot, a cubic nanoparticle, a cuboctahedron nanoparticle, a spherical nanoparticle, a pseudo-spherical nanoparticle, a faceted nanoparticle, a nanorod, a nanowire, a tetrapod, a branched nanoparticle, or other suitable particle having a dimension on the nanometer scale. The pores 710 allow gas to transfer from outside the metal oxide shell 702 to a surface 712 of the nanoparticle 704. In an embodiment, the shell is a mesoporous shell, which is a shell that includes pores having a diameter (or cross-sectional size) within a range of 2 to 50 nm. For example, the mesoporous shell may include pores in which a majority has a cross-sectional size within the range and including sub-1 nm to 4 nm.

In an embodiment, the metal-oxide shell 702 includes a first metal oxide selected from SiO₂, Al₂O₃, TiO₂, ZrO₂, Ta₂O₅, Nb₂O₅, their binary, ternary mixed oxides, or some other suitable metal oxide. In a particular embodiment, the metal-oxide shell includes SiO₂. In an embodiment, the nanoparticle is a metal nanoparticle. In an embodiment, the metal nanoparticle includes a first metal selected from Pt, Pd, Ru, Rh, Ir, Os, Au, Ag, Cu, Ni, Co, Fe, or their binary, ternary combinations, or some other suitable metal. In a particular embodiment, the metal nanoparticle is a Pt nanoparticle. Applications of the core-shell nanoparticle include catalysis in which the core-shell nanoparticle is a nanocatalyst.

An embodiment of a method of making a core-shell nanoparticle 700 of the present invention includes forming a metal-oxide shell on a colloidal nanoparticle. The colloidal nanoparticle includes a nanoparticle and a capping agent on the surface of the nanoparticle. In an embodiment, the capping agent includes a first capping agent that is selected from TTAB (tetradecyltrimethylammonum bromide), CTAB (cetyltrimethylammonium bromide), alkyl ammonium halide, alkyl amine, alkyl thiol, alkyl phosphine, PVP (poly(vinylpyrrolidone)), and some other suitable capping agent (e.g., a surfactant or polymeric capping agent). In an embodiment, forming the metal-oxide shell employs a polymerization process (e.g., a sol-gel polymerization process). Forming the metal-oxide shell on the colloidal nanoparticle produces a precursor core-shell nanoparticle. The capping agent is removed from the precursor core-shell nanoparticle to produce the core-shell nanoparticle 700. In an embodiment, removing the capping agent employs a calcination process that includes heating the precursor core-shell nanoparticle to at least 300° C. in an O₂ containing environment (e.g. heating in air).

It will be readily apparent to one skilled in the art that capping agents are also referred to as ligands.

An embodiment of method of using a nanocatalyst of the present invention includes providing a core-shell nanoparticle 700 (i.e. the nanocatalyst). Reactants are introduced into a vicinity of the nanocatalyst, which produces a reaction of the reactants that is facilitated or enhanced by the nanocatalyst. Reactions that may be produced include oxidation, partial oxidation, hydrocarbon cracking, combustion, hydrogenation, and other suitable reactions. In an embodiment, the core-shell nanoparticle 700 includes a Pt core and a mesoporous SiO₂ shell, which may be used as a nanocatalyst for oxidation of CO or hydrogenation of ethylene.

Discussion:

We report the design of a high-temperature stable model catalytic system that includes a Pt metal core coated with a mesoporous silica shell (Pt@mSiO₂). While inorganic silica shells encaged the Pt cores up to 750° C. in air, the mesopores directly accessible to Pt cores made the Pt@mSiO₂ nanoparticles as catalytically active as bare Pt metal for ethylene hydrogenation and CO oxidation. The high thermal stability of Pt@mSiO₂ nanoparticles permitted high-temperature CO oxidation studies, including ignition behavior, which was not possible for bare Pt nanoparticles because of their deformation or aggregation. The results suggest that the Pt@mSiO₂ nanoparticles are excellent nanocatalytic systems for high-temperature catalytic reactions or surface chemical processes, and the design concept employed in the Pt@mSiO₂ core-shell catalyst can be extended to other metal-metal oxide compositions.

In this work, we designed core-shell particle configurations and prepared the nanoparticles with high thermal stability. The core-shell structures have important implications in catalysis (ref 29). The outer shells isolate the catalytically active nanoparticle cores and prevent the possibility of sintering of core particles during catalytic reactions at high temperatures. Additionally, the synergistic effects of metal-support interfaces may be maximized where such interfaces are important in catalytic performances.

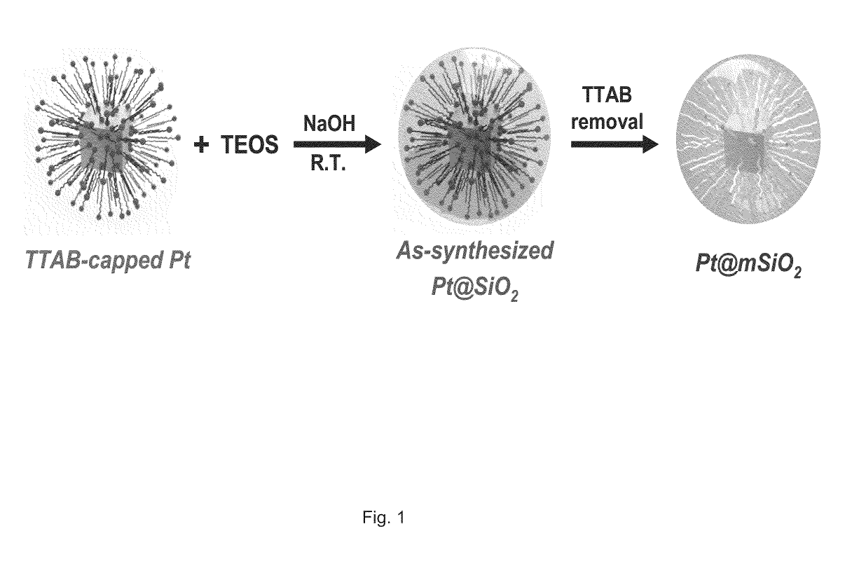

We prepared Pt-mesoporous silica core-shell (Pt@mSiO₂) nanoparticles that are thermally stable at high temperatures and performed CO oxidation and ethylene hydrogenation reactions to explore the catalytic activities of the Pt@mSiO₂ core-shell nanoparticles. The core-shell structured Pt@mSiO₂ nanoparticles were prepared in three steps (FIG. 1): (i) synthesis of Pt nanoparticles using tetradecyltrimethylammonum bromide (TTAB) as the capping agent, (ii) silica polymerization around the Pt cores generating the as-synthesized Pt@SiO₂ mesostructures and (iii) removal of the TTAB molecules by calcination to produce the Pt@mSiO₂ core-shell nanoparticles. The Pt@mSiO₂ consisted of 14 nm Pt cores and 17 nm thick mesoporous silica shells. The reacting molecules are directly accessible to the Pt cores through the mesopores within the silica shells and the product molecules can readily exit through these mesopores. The Pt cores were encaged within the silica shells at temperatures up to 750° C. in air. The Pt@mSiO₂ nanoparticle catalysts exhibited catalytic activity similar to TTAB-capped Pt nanoparticles for ethylene hydrogenation and CO oxidation. Significantly, the high thermal stability of Pt@mSiO₂ nanoparticles enabled the study of ignition behavior during the Pt nanoparticle catalyzed CO oxidation process. The high thermal stability, as well as uniform mesoporous shell structure, suggested that the Pt@mSiO₂ core-shell nanoparticles are an excellent nanoparticle system for catalytic reactions or surface chemical processes that occur at high temperatures.

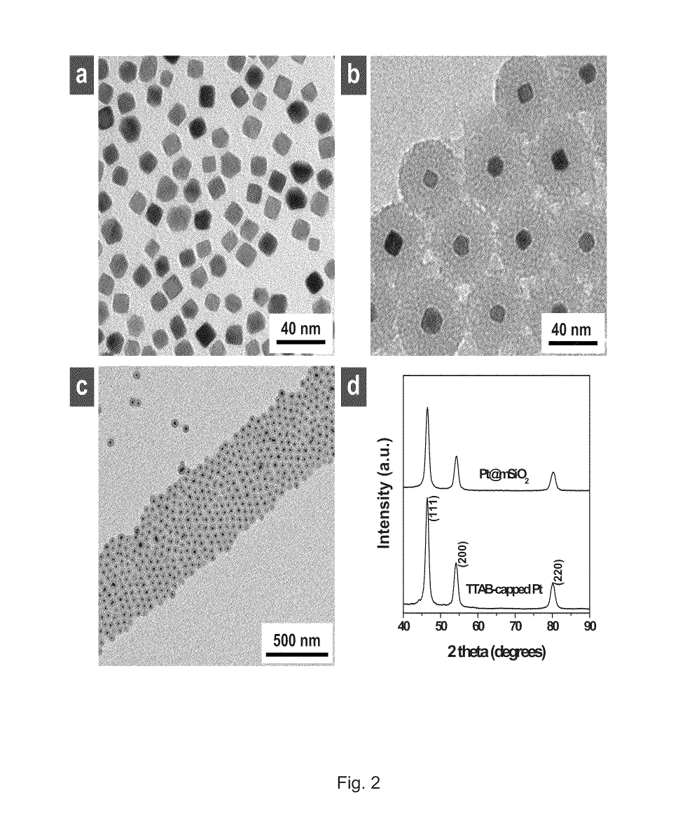

TEM images for TTAB-capped Pt and as-synthesized Pt@SiO₂ nanoparticles are provided in FIG. 2. The TTAB-capped Pt nanoparticles displayed in FIG. 2a were composed of a mixture of cubes (70%), cuboctahedra (26%) and irregular shapes (4%) and exhibited an average particle size around 14.3 nm in diagonal distance. The surfaces of these Pt cubes are stabilized by the bilayer of the TTAB surfactants (ref. 30). These surface capping TTAB surfactants were also used as structure directing templates for the polymerization of silicates by a sol-gel process, as demonstrated in the synthesis of MCM-41-like ordered mesoporous silicas (refs. 31, 32). The silica-TTAB layer was formed around the Pt nanoparticles under basic conditions (pH=10-11) through an electrostatic interaction between the cationic (TTAB) and anionic (silicate) species. The TEM images of as-synthesized Pt@SiO₂ obtained under optimized experimental conditions (Pt colloid:TEOS=1:4.5), where each Pt particle is encapsulated by a silica layer, are shown in FIGS. 1b and 1c. The average thickness of the silica layer surrounding the Pt core was around 17 nm. FIG. 2c displays the closely assembled structure of the Pt@SiO₂ nanoparticles in a large area, which was formed by drop casting on a TEM grid. The XRD patterns for Pt and Pt@SiO₂ nanoparticles (FIG. 2d) revealed that the crystal structure (face centered cubic) TTAB-capped Pt nanoparticles was maintained after the formation of the silica layer on Pt.

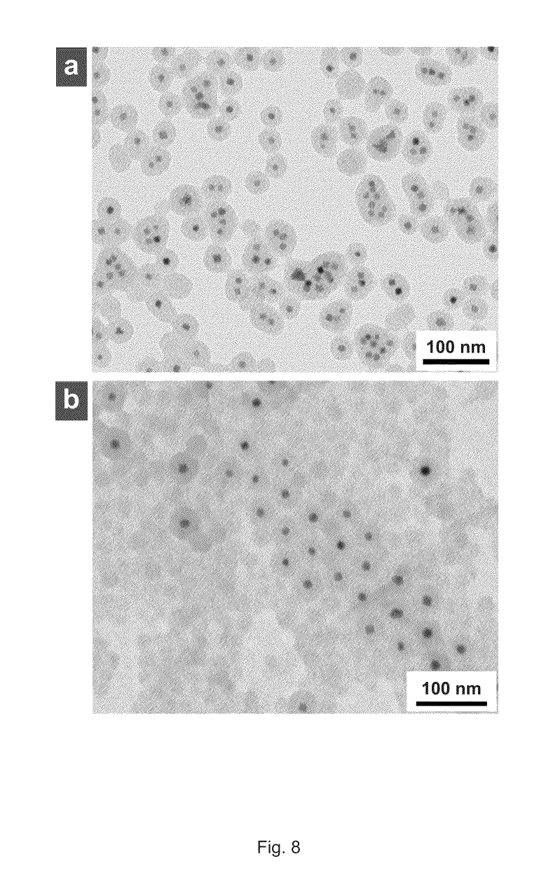

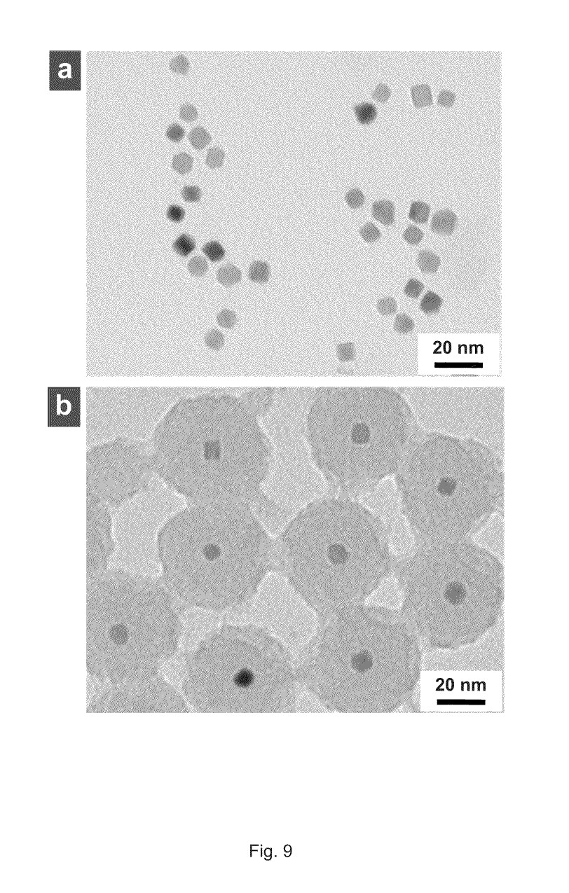

Most (˜95%) of the as-synthesized Pt@SiO₂ nanoparticles shown in FIGS. 2b and 2c exhibited the core-shell structures, where each Pt particle is encaged within a silica shell. The configuration of core-shell nanoparticles was tuned by changing the concentration of the silica source (TEOS) added for the silica polymerization. If the concentration of TEOS was decreased compared to the amount required for optimized condition, the silica shell contained multiple Pt particles (FIG. 8a). Increasing the amount of TEOS resulted in the formation of a mixture of Pt@mSiO₂ and amorphous silica particles without Pt cores (FIG. 8b). This synthetic strategy can be generally applicable to nanoparticles with differing composition, size and shape. For instance, smaller size 8.5 nm TTAB-capped Pt nanoparticles can be readily converted into Pt@SiO₂ core-shell particles, as shown in FIG. 9.

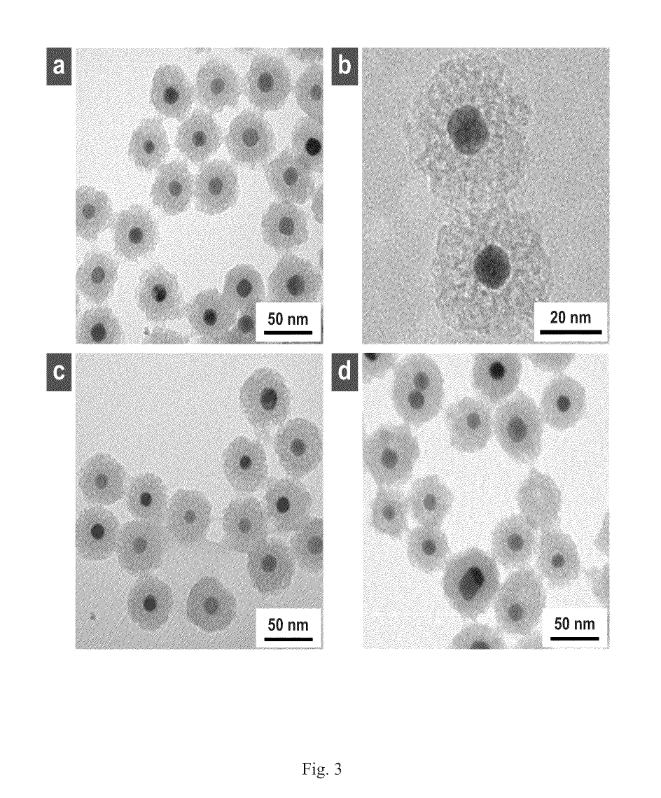

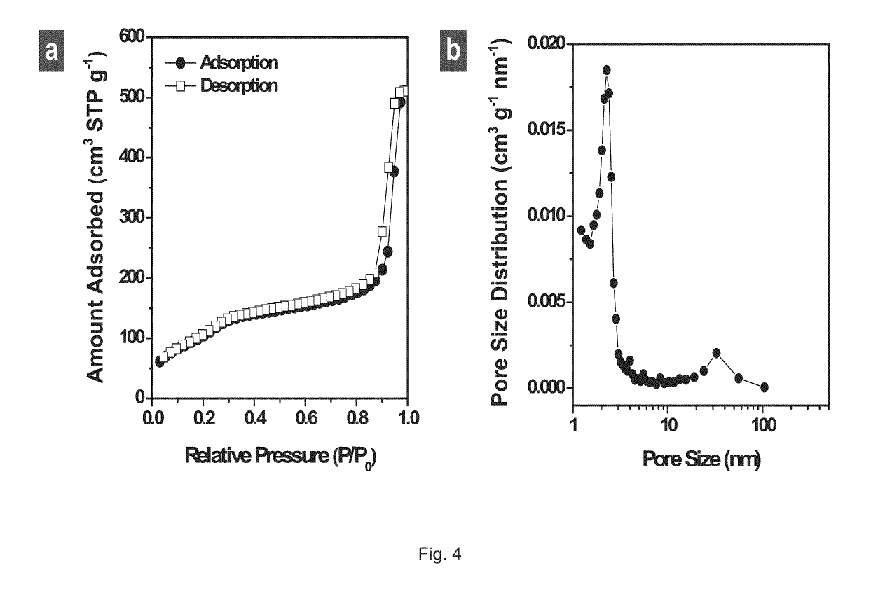

The as-synthesized Pt@SiO₂ nanoparticles contained a significant amount of the TTAB surfactants that are unfavorable for reactant and product molecular diffusion in catalytic applications. To remove the TTAB surfactants, the as-synthesized Pt@SiO₂ sample was calcined at 350° C. for 2 h in static air to yield mesoporous Pt@mSiO₂ nanoparticles. The TEM images of Pt@mSiO₂ nanoparticles after calcination (FIGS. 3a and 3b) exhibited mesopores of 2-3 nm in the silica shells. The porosity of Pt@mSiO₂ was confirmed by nitrogen physisorption. The nitrogen adsorption-desorption isotherms of Pt@mSiO₂ calcined at 350° C. (FIG. 4a) revealed that these nanoparticles are mesoporous, as identified by the increase of the adsorption amount in the relative pressure (P/P₀) range of 0.2 to 0.3. The pore size distribution curve calculated from adsorption branch of isotherms (FIG. 4b) exhibited a maximum at 2.3 nm and the BET surface area of Pt@mSiO₂ was calculated to be 440 m² g⁻¹, indicating the highly mesoporous nature of the silica shell.

For core-shell nanoparticles to be catalytically active, direct access of reactive molecules to the core particles is of significant importance. In our design of these core-shell particles, the mesoporous silica layer was directly formed on the Pt cores with pores extending through the silica layer. The accessibility of gas molecules was directly proven by chemisorption measurements. Hydrogen chemisorption over the Pt@mSiO₂ catalyst gave a dispersion value of 8±0.5%, which is comparable with the ratio of surface atoms (11%) on the Pt particle, as calculated by geometric considerations (ref 36).

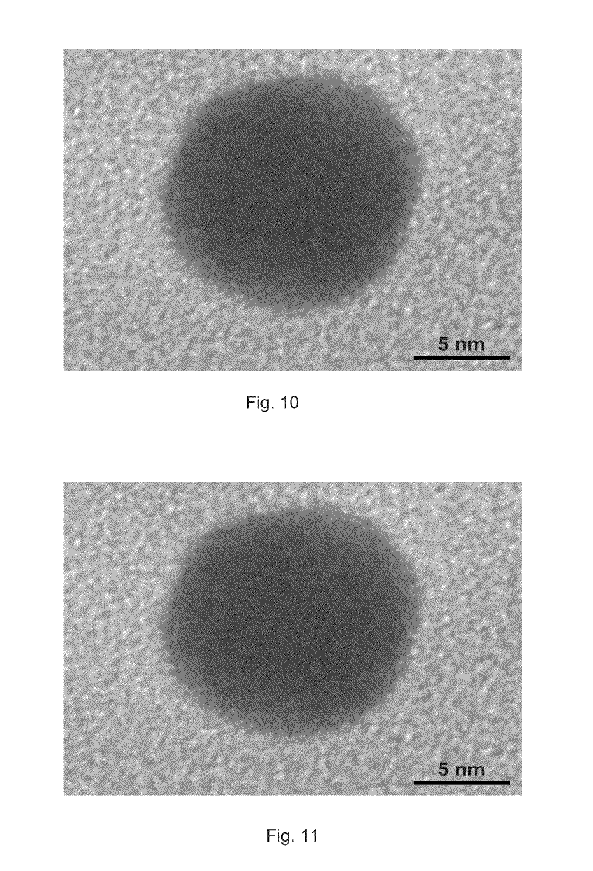

After calcination at 350° C., the spherical core-shell shape of the as-synthesized Pt@SiO₂ particles was maintained, as can be seen by comparing the TEM images before (FIG. 2b) and after (FIG. 3a) calcination. The shape of the Pt cores inside Pt@mSiO₂ became slightly rounder after calcination, which is presumably due to the melting of the Pt particles at a Pt-silica interface and which appears to be consistent with an earlier report that looked at Pt nanoparticles resting on a silica surface (ref 37). However, a closer observation of the Pt core by high resolution TEM image in FIG. 10 revealed that the Pt core continued to be facetted and single crystalline after thermal treatment under air. The stability of Pt@mSiO₂ core-shell nanoparticles at higher temperatures was investigated by heating the sample to 550° C. and 750° C. in air. After the calcination at 550° C. (FIG. 3c), the core-shell morphology of Pt@mSiO₂ nanoparticles was preserved. Even upon heating the sample as high as 750° C., the morphology of most Pt@mSiO₂ nanoparticles was maintained and the Pt cores were still encaged within the silica shells (FIG. 3d), indicating high thermal stability of Pt@mSiO₂ core-shell nanoparticles. It is interesting to note that in the case of sample calcined at 750° C., some of core-shell particles possessing larger Pt cores and Pt-free hollow silica particles were found. It seemed that Pt cores in Pt@mSiO₂ nanoparticles diffused around 750° C. through the mesoporous silica shell into the neighboring Pt@mSiO₂ particles. A detailed study of the thermal evolution of Pt@mSiO₂ nanoparticles is currently being investigated.

Our present design of Pt@mSiO₂ core-shell nanoparticles allows the direct access of reactive molecules to the catalytically active core metals. In addition, the Pt cores within the silica layer can be encaged even after high-temperature treatments while the faceted nature of the particle is preserved, showing great promise for use in high-temperature catalytic reactions.

The catalytic activity of Pt@mSiO₂ nanoparticles was investigated in ethylene hydrogenation. The ethylene hydrogenation was performed at 10 Torr of ethylene, 100 Torr of H₂, with the balance He (see Supplementary Information below for experimental details). The Pt@mSiO₂ exhibited a TOF of 6.9 s⁻¹ at 25° C. and activation energy (Eₐ) of 8.1 kcal mol⁻¹. The TOF and activation energy are similar to those of the Pt single crystal, colloidal Pt nanoparticle loaded SBA-15 model catalysts, and other supported catalysts (see Table 1 in Supplementary Information below). It is worth noting that the Pt@mSiO₂ nanoparticles exhibited an order of magnitude higher TOF than the Pt@CoO yolk-shell nanoparticles (ref. 38). The higher activity of Pt@mSiO₂ is likely due to the more facile diffusion and transport of the reactants and products through the mesoporous silica shells in Pt@mSiO₂ than the CoO shell in Pt@CoO where the grain boundaries in CoO were proposed as entry points for the molecules (ref. 38).

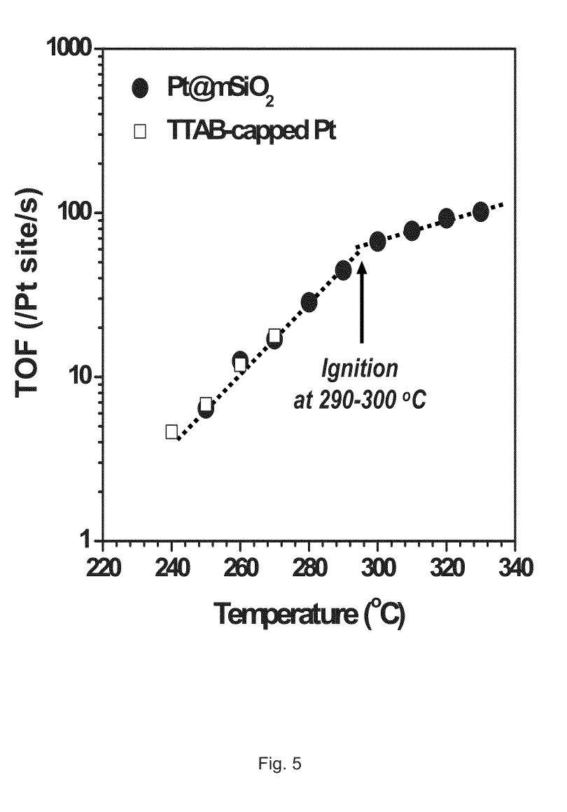

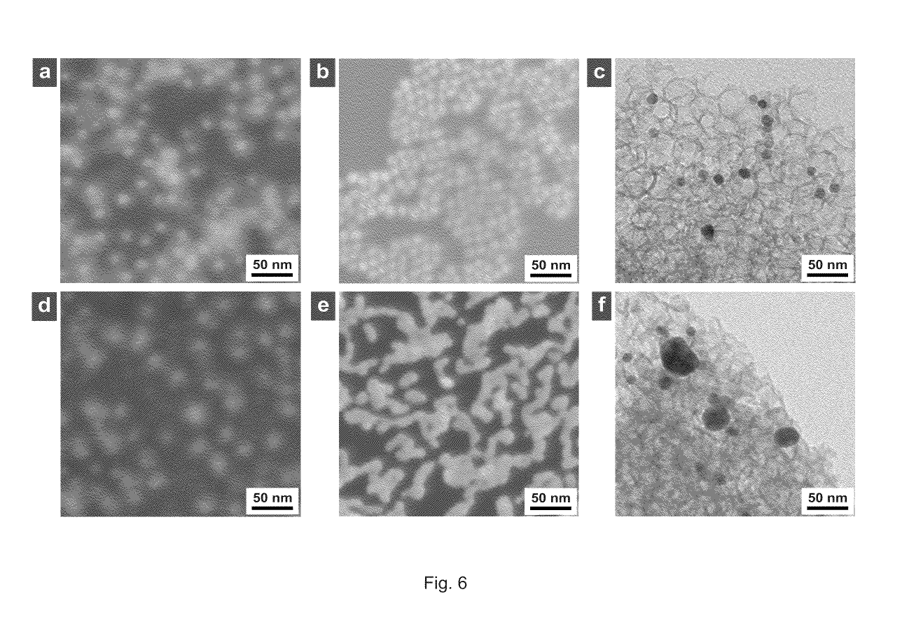

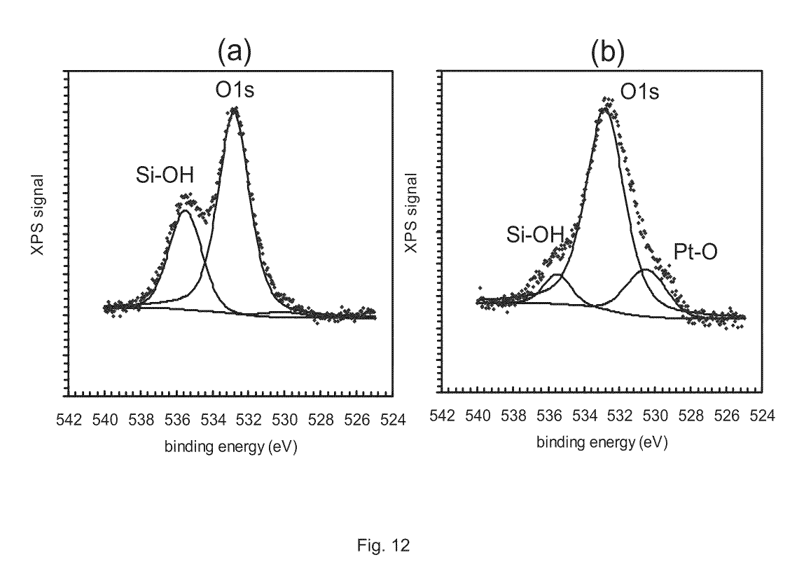

The high-temperature catalytic properties of Pt@mSiO₂ core-shell nanoparticles were explored using CO oxidation as a model reaction. The catalytic oxidation of CO to CO₂ over platinum group metals has been one of the most widely studied surface reactions due to its significance for emission control and fuel cells (refs. 20-25). In particular, from the mechanistic point of view, the CO oxidation reaction is intriguing as the reaction proceeds via different mechanisms below and above the ignition temperature. For the CO oxidation reaction, the Pt@mSiO₂ and Pt nanoparticles were deposited on a silicon wafer using the Langmuir-Blodgett (LB) technique. CO oxidation was performed with excess O₂ (40 Torr CO, 100 Torr O₂, with the balance He) in the temperature range of 240 to 340° C. FIG. 5 presents the activity of the Pt@mSiO₂. Two distinct reaction regimes are observed as a function of temperature, which indicated an ignition temperature around 290-300° C. For comparison, the TTAB-capped Pt nanoparticle catalyst on the silicon wafer was also tested for CO oxidation. The activity of the Pt@mSiO₂ catalyst was as high as TTAB-capped Pt nanoparticles, indicating that the silica shells in the Pt@mSiO₂ nanoparticles were porous enough to provide access to the Pt cores, which is consistent with our chemisorption and TEM studies. FIG. 6 comparatively displays SEM and TEM images of TTAB-capped Pt nanoparticle arrays on the silicon wafer and Pt dispersed inside the mesopores of MCF mesoporous silica (Pt/MCF), as well as the core-shell Pt@mSiO₂ before and after CO oxidation at 300° C. The Pt@mSiO₂ catalyst after CO oxidation at 330° C. maintained the morphology of calcined particles (FIG. 6b). More importantly, the faceted and crystalline nature of the Pt cores in the Pt@mSiO₂ catalyst was preserved after the reaction, as shown in FIG. 11. However, the TTAB-capped Pt on silicon wafer (FIG. 6d) and Pt/MCF (FIG. 6f) exhibited severe aggregation of Pt particles after CO oxidation at 300° C., which hampered the quantitative study of CO oxidation above the ignition temperature regime. The XPS spectra of O1s peaks of Pt@mSiO₂ revealed that the Pt core was partially oxidized after CO oxidation (FIG. 12).

The ignition temperature during CO oxidation over the Pt@mSiO₂ catalyst is 290-300° C., which lies between that of Pt (100) (227° C.) and Pt (111) (347° C.) single crystals (ref. 25). The Pt cores encaged in Pt@mSiO₂ nanoparticles are mostly composed of cubic and cuboctahedron shapes, exposing mostly (100) and (111) surfaces, which explains the reason for the ignition temperature of the Pt@mSiO₂ nanoparticles to be between those of Pt (100) and Pt (111) single crystals. The Pt@mSiO₂ nanoparticles exhibited lower activation energies (27.5 and 9.8 kcal mol⁻¹ for below and above ignition temperature, respectively) than Pt (111) single crystal (42 and 14 kcal mol⁻¹)²⁴ and (100) single crystal (32.9 kcal mol⁻¹ for below ignition) (ref. 22). For catalytic reactions on the surface to occur, the reacting molecules, reaction intermediates and products must alter their bond distances to allow rapid rearrangement. A relatively small number of bonds must also be broken and reformed as the catalytic chemistry occurs. The chemical bonds rearrange more easily on nanoparticles, where fewer atoms participate in the restructuring during catalytic turnover than on single crystal surfaces and this phenomena might be responsible for the origin of lower activation energies.

In summary, the core-shell structured Pt-mesoporous silica (Pt@mSiO₂) nanoparticles were designed as high-temperature model catalysts. The Pt@mSiO₂ nanoparticles maintained their core-shell configurations up to 750° C. and exhibited high catalytic activity for ethylene hydrogenation and CO oxidation. The mesoporous silica coating chemistry on nanoparticle surface is straightforward. Thus, the method can potentially be extended to other nanoparticle cores with different composition, size, and shape and to other shell compositions. The CO oxidation study highlights the role of the thermally stable inorganic silica shell in the Pt@mSiO₂ nanoparticles that permit the study of catalytic reactions or surface phenomena taking place at high temperatures. Further application of core-shell catalysts to high-temperature reactions, such as partial oxidation and cracking of hydrocarbon and catalytic combustion, appears possible.

Methods:

Synthesis of TTAB-capped Pt and Pt@mSiO₂ core-shell nanoparticles: The synthesis of TTAB-capped Pt nanoparticles was performed by following the reported method with a modification (ref 30). The detailed synthesis procedure for Pt nanoparticles has been described in the Supplementary Information. The Pt@mSiO₂ core-shell nanoparticles were prepared by polymerizing the silica layer around the surface of Pt nanoparticles via a sol-gel process (refs. 33-35, 44-46). The Pt nanoparticle colloid (4.5×10⁻⁵ mole) dispersed in 5 mL of DI water was added to 35.5 mL of DI water. A NaOH solution (1.0 mL of 0.05 M) was added to the aqueous Pt colloid solution with stirring to adjust the pH of the solution to around 10-11. To this basic solution, a controlled amount of 10 vol % tetraethylorthosilicate (TEOS) diluted with methanol was added to initiate the silica polymerization. The as-synthesized Pt@SiO₂ was calcined at 350° C. or higher for 2 h in static air to remove TTAB surfactants to generate Pt@mSiO₂ particles. The 2D model catalyst systems were fabricated by depositing the colloidal Pt and Pt@SiO₂ nanoparticles on a silicon wafer using the LB technique (see Supplementary Information). MCF mesoporous silica with large mesopores, around 30 nm, was synthesized following the method found in the literature (ref 47) and TTAB-capped Pt nanoparticle was incorporated inside the pores of the MCF silica by capillary inclusion (ref. 48) to produce the 3D model catalyst.

Characterization: The particle size and shape were analyzed with transmission electron microscope (TEM) images using a Philips/FEI Tecnai 12 microscope operating at 100 kV and an FEI Tecnai G² S-Twin electron microscope operating at 200 kV. X-ray diffraction (XRD) patterns were measured on a Bruker D8 GADDS diffractometer using Co K radiation (1.79 Å). Nitrogen physisorption experiments were performed using a Quantachrome Autosorb-1 analyzer at −196° C. Before the measurement, degassing was conducted at 200° C. for 12 h to remove possible moisture. Hydrogen chemisorption was also carried out with a Quantachrome Autosorb-1 at 30° C. Before the chemisorptions, the sample was heated to 300° C. for 1 h under H₂ and evacuated at 310° C. for 1.5 h, then cooled down to room temperature. The morphology and chemical composition of the 2-D LB films were characterized with a scanning electron microscope (SEM) and X-ray photoelectron spectroscopy (XPS), respectively. SEM images were taken on a Zeiss Gemini Ultra-55 with a beam energy of 5 kV. XPS spectra were taken on a 15 kV, 350 Watt PHI 5400 ESCA/XPS system equipped with an Al anode X-ray source.

CO oxidation measurements: CO oxidation studies were performed in an ultrahigh vacuum chamber with a base pressure of 5.0×10⁻⁸ Torr (ref. 19). The reactions were carried out under excess O₂ conditions: 40 Torr CO, 100 Torr O₂, and 620 Torr He. The gases were circulated through the reaction line by a Metal Bellows recirculation pump at a rate of 2 L min⁻¹. The volume of the reaction loop is 1.0 L. An HP Series II gas chromatograph equipped with a thermal conductivity detector and a 15′, ⅛″ SS 60/80 Carboxen-1000 (Supelco) was used to separate the products for analysis. The measured reaction rates are reported as turnover frequencies (TOF) and are measured in units of product molecules of CO₂ produced per metal surface site per second of reaction time. The number of metal sites is calculated by geometrical considerations based on SEM measurements of the surface area of a nanoparticle array.

Supplementary Information (Experimental Details):

Synthesis of TTAB-capped Pt nanoparticles: For the synthesis of Pt nanoparticles, 5 mL of aqueous 10 mM K₂PtCl₄ (Aldrich, 99.9%) and 12.5 mL of 400 mM TTAB (Aldrich, 99%) were mixed with 29.5 mL of deionized water (DI) in a 100-mL round bottom flask at room temperature. The mixture was stirred at room temperature for 10 min and was heated at 50° C. for 10 min. To the clear solution, 3 mL of 500 mM ice-cooled NaBH₄ (Aldrich, 98%) solution was injected through the septum using a syringe. The gas evolved inside the flask was released by inserting a needle through the septum for 20 min. The needle was then removed and the solution was kept at 50° C. for 15 h. The product was centrifuged at 3000 rpm for 30 min. The supernatant solution was separated and centrifuged again at 14000 rpm for 15 min, twice. The Pt nanoparticle colloids were collected and re-dispersed in 5 mL of deionized water by sonication for further use.

Fabrication of Langmuir-Blodgett films of Pt and Pt@SiO₂ nanoparticles: Colloidal Pt or Pt@SiO₂ nanoparticle solutions were dispersed on the surface of DI subphase on a LB trough (type 611, NIMA Technology) at room temperature. The surface pressure was monitored with a Wilhelmy plate and adjusted to zero before spreading the nanoparticles. The resulting surface layer was compressed by a mobile barrier at a rate of 20 cm² min⁻¹. The nanoparticles were deposited by lifting up the silicon substrates (which had been immersed in water subphase before the nanoparticles were dispersed) at a surface pressure of ˜12 mN m⁻¹.

Measurement of ethylene hydrogenation: The ethylene hydrogenation was conducted in a plug flow reactor made of Pyrex. Reactants and products were detected by gas chromatography (Hewlett-Packard 5890 Series II). Rate measurement for ethylene hydrogenation was conducted at differential conditions (all conversions<10%).

Table 1 provides a comparison of ethylene hydrogenation activity of Pt@mSiO₂ nanoparticles (last row in Table 1) versus single crystal and supported catalysts (first seven rows in Table 1) in which reaction conditions were 10 Torr C₂H₄, 100 Torr H₂, and 298 K.

TABLE 1

| Catalyst | Eₐ (kcal mol⁻¹) | TOF (s⁻¹) | Ref. |
|---|---|---|---|
| Pt(111) | 10.8 | 9.3 | (ref. 49) |
| Pt nanoparticle array | 10.2 | 14.3 | (ref. 50) |
| 0.5% Pt/SiO₂ | 8.9 | 17.5 | (ref. 51) |
| 0.04% Pt/SiO₂ | 8.6 | 4.4 | (ref. 52) |
| ~1% Pt/SBA-15 by | 7 | ~0.7 | (ref. 53) |
| capillary inclusion | | | |
| ~1% Pt/SBA-15 by | 10~12 | ~3.5 | (ref. 14) |
| nanoencapsulation | | | |
| 14% Pt@CoO | 5.0 | 0.29 | (ref. 54) |
| Pt@mSiO₂ | 8.1 | 6.9 | This Work |

### Additional Examples

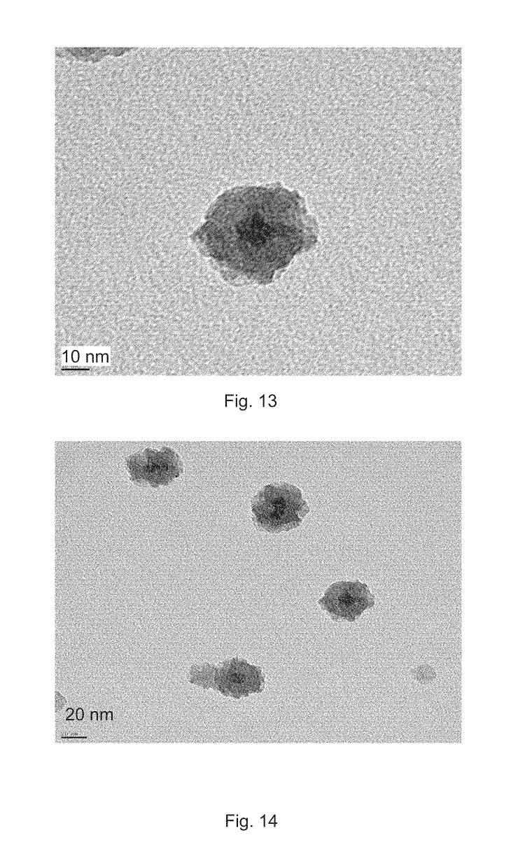

FIGS. 13 and 14 provide TEM images of example of core-shell nanoparticles of the present invention in which the core is a Co nanoparticle and the shell is a mesoporous SiO₂ shell. These core-shell nanoparticles were prepared using a process similar to the process used to prepare the example core-shell nanoparticles that included a Pt core and a mesoporous SiO₂ shell with the exception that the core nanoparticles were Co nanoparticles.

### REFERENCES

1. Bell, A. T. The impact of nanoscience on heterogeneous catalysis. Science 299, 1688-1691 (2003).
2. Schlögl, R. & Abd Hamid, S. B. Nanocatalysis: mature science revisited or something really new? Angew. Chem. Int. Ed. 43, 1628-1637 (2004).
3. Somorjai, G. A., Contreras, A. M., Montano, M. & Rioux, R. M. Clusters, surfaces, and catalysis. Proc. Natl. Acad. Sci. USA 103, 10577-10583 (2006).
4. Thomas, J. M. Heterogeneous catalysis: enigmas, illusions, challenges, realities, and emergent strategies of design. J. Chem. Phys. 128, 182502 (2008).
5. Somorjai, G. A. Introduction to Surface Chemistry and Catalysis(Wiley, New York, 1994).
6. Over, H. et al. Atomic-scale structure and catalytic reactivity of the RuO₂(110) surface. Science 287, 1474-1476 (2000).
7. Greeley, J. & Mavrikakis, M. Alloy catalysts designed from first principles. Nat. Mater. 3, 810-815 (2004).
8. Stamenkovic, V. R. et al. Improved oxygen reduction activity on Pt₃Ni(111) via increased surface site availability. Science 315, 493-497 (2007).
9. Roucoux, A., Schulz, J. & Patin, H. Reduced transition metal colloids: a novel family of reusable catalysts? Chem. Rev. 102, 3757-3778 (2002).
10. Park, J., Joo, J., Kwon, S. G., Jang, Y. & Hyeon, T. Synthesis of monodisperse spherical nanocrystals. Angew. Chem. Int. Ed. 46, 4630-4660 (2007).
11. Tao, A. R., Habas, S. & Yang, P. Shape control of colloidal metal nanocrystals. Small 4, 310-325 (2008).
12. Ahmadi, T. S., Wang, Z. L., Green, T. C., Henglein, A. & El-Sayed, M. A. Shape-controlled synthesis of colloidal platinum nanoparticles. Science 272, 1924-1926 (1996).
13. Sun, Y. & Xia, Y. Shape-controlled synthesis of gold and silver nanoparticles. Science 298, 2176-2179 (2002).
14. Song, H. et al. Hydrothermal growth of mesoporous SBA-15 silica in the presence of PVP-stabilized Pt nanoparticles: synthesis, characterization, and catalytic properties. J. Am. Chem. Soc. 128, 3027-3037 (2006).
15. Narayanan, R. & El-Sayed, M. A. Shape-dependent catalytic activity of platinum nanoparticles in colloidal solution. Nano Lett. 4, 1343-1348 (2004).
16. Tian, N., Zou, Z.-Y., Sun, S.-G., Ding, Y. & Wang, Z. L. Synthesis of tetrahexahedral platinum nanocrystals with high-index facets and high electro-oxidation activity. Science 316, 732-735 (2007).
17. Bratlie, K. M., Lee, H., Komvopoulos, K., Yang, P. & Somorjai, G. A. Platinum nanoparticle shape effects on benzene hydrogenation selectivity. Nano Lett. 7, 3097-3101 (2007).
18. Alayoglu, S., Nilekar, A. U., Mavrikakis, M. & Eichhorn, B. Ru—Pt core-shell nanoparticles for preferential oxidation of carbon monoxide in hydrogen. Nat. Mater. 7, 333-338 (2008).
19. Park, J. Y., Zhang, Y., Grass, M., Zhang, T. & Somorjai, G. A. Tuning of catalytic CO oxidation by changing composition of Rh—Pt bimetallic nanoparticles. Nano Lett. 8, 673-677 (2008).
20. Langmuir, I. The mechanism of the catalytic action of platinum in the reactions 2CO+O₂=2CO₂ and 2H₂+O₂=2H₂O. Trans. Frad. Soc. 621-654 (1922).
21. Campbell, C. T., Ertl, G., Kuipers, H. & Segner, J. A molecular beam study of the catalytic oxidation of CO on a Pt(111) surface. J. Chem. Phys. 73, 5862-5873 (1980).
22. Berlowitz, P. J., Peden, C. H. F. & Goodman, D. W. Kinetics of CO oxidation on single-crystal Pd, Pt, and Ir. J. Phys. Chem. 92, 5213-5221 (1988).
23. Chen, M. S. et al. Highly active surfaces for CO oxidation on Rh, Pd, and Pt. Surf Sci. 601, 5326-5331 (2007).
24. Su, X. C., Cremer, P. S., Shen, Y. R. & Somorjai, G. A. High-pressure CO oxidation on Pt(111) monitored with infrared-visible sum frequency generation (SFG). J. Am. Chem. Soc. 119, 3994-4000 (1997).
25. McCrea, K. R., Parker, J. S. & Somorjai, G. A. The role of carbon deposition from CO dissociation on platinum crystal surfaces during catalytic CO oxidation: effects on turnover rate, ignition temperature, and vibrational spectra. J. Phys. Chem. 106, 10854-10863 (2002).
26. Zaera, F. The surface chemistry of hydrocarbon partial oxidation catalysis. Catal. Today 81, 149-157 (2003).
27. Huber, G. W. & Corma, A. Synergies between bio- and oil refineries for the production of fuels from biomass. Angew. Chem. Int. Ed. 46, 7184-7201 (2007).
28. Ciuparu, D., Lyubovsky, M. R., Altman, E., Pfefferle, L. D. & Datye, A. Catalytic combustion of methane over palladium-based catalysts. Catal. Rev. 44, 593-649 (2002).
29. Somorjai, G. A. & Rioux, R. M. High technology catalysts towards 100% selectivity. Fabrication, characterization and reaction studies. Catal. Today 100, 201-215 (2005).
30. Lee, H. et al. Morphological control of catalytically active platinum nanocrystals. Angew. Chem. Int. Ed. 45, 7824-7828 (2006).
31. Kresge, C. T., Leonowicz, M. E., Roth, W. J., Vartuli, J. C. & Beck, J. S. Ordered mesoporous molecular sieves synthesized by a liquid-crystal mechanism. Nature 359, 710-712 (1992).
32. Corma, A. From microporous to mesoporous molecular sieve materials and their use in catalysis. Chem. Rev. 97, 2373-2419 (1997).
33. Kim, M., Sohn, K., Na, H. B. & Hyeon, T. Synthesis of nanorattles composed of gold nanoparticles encapsulated in mesoporous carbon and polymer shells. Nano Lett. 2, 1383-1387 (2002).
34. Nooney, R. I., Thirunavukkarasu, D., Chen, Y., Josephs, R. & Ostafin, A. E. Self-assembly of mesoporous nanoscale silica/gold composites Langmuir 19, 7628-7637 (2003).
35. Botella, P., Corma, A. & Navarro, M. T. Single gold nanoparticles encapsulated in monodispersed regular spheres of mesostructured silica produced by pseudomorphic transformation. Chem. Mater. 19, 1979-1983 (2007).
36. van Hardeveld, R. & Hartog, F. The statistics of surface atoms and surface sites on metal crystals. Surf. Sci. 15, 189-230 (1969).
37. Yu, R., Song, H., Zhang, X.-F. & Yang, P. Thermal wetting of platinum nanocrystals on silica surface. J. Phys. Chem. B 109, 6940-6943 (2005).
38. Yin, Y. et al. Formation of hollow nanocrystals through the formation of nanoscale Kirkendall effect. Science 304, 711-714 (2004).
39. Lin, K.-J., Chen, L.-J., Prasad, M. R. & Cheng, C. Y. Core-shell synthesis of a novel, spherical, mesoporous silica/platinum nanocomposite: Pt/PVP@MCM-41. Adv. Mater. 16, 1845-1849 (2004).
40. Ikeda, S. et al. Ligand-free platinum nanoparticles encapsulated in a hollow porous carbon shell as a highly active heterogeneous hydrogenation catalyst. Angew. Chem. Int. Ed. 45, 7063-7066 (2006).

41. Arnal, P. M., Comotti, M. & Schüth, F. High-temperature-stable catalysts by hollow sphere encapsulation. Angew. Chem. Int. Ed. 45, 8224-8227 (2006).
42. Lee, J., Park, J. C. & Song, H. A nanoreactor framework of a Au@SiO₂ yolk/shell structure for catalytic reduction of p-nitrophenol. Adv. Mater. 20, 1523-1528 (2008).
43. Yu, K., Wu, Z., Zhao, Q., Li, B. & Xie, Y. High-temperature-stable Au@SnO₂ core/shell supported catalyst for CO oxidation. J. Phys. Chem. C 112, 2244-2247 (2008).
44. Liz-Marzan, L. M., Giersig, M. & Mulvaney, P. Synthesis of nanosized gold-silica core-shell particles. Langmuir 12, 4329-4335 (1996).
45. Lu, Y., Yin, Y., Li, Z.-Y. & Xia, Y. Synthesis and self-assembly of Au@SiO₂ core-shell colloids. Nano Lett. 2, 785-788 (2002).
46. Gorelikov, I. & Matsuura, N. Single-step coating of mesoporous silica on cetyltrimethyl ammonium bromide-capped nanoparticles. Nano Lett. 8, 369-373 (2008).
47. Schmidt-Winkel, P. et al. Microemulsion templating of siliceous mesostructured cellular foams with well-defined ultralarge mesopores. Chem. Mater. 12, 686-696 (2000).
48. Rioux, R. M., Song, H., Hoefelmeyer, J. D., Yang, P. & Somorjai, G. A. High-surface-area catalyst design: synthesis, characterization, and reaction studies of platinum nanoparticles in mesoporous SBA-15 silica. J. Phys. Chem. B 109, 2192-2202 (2005).
49. Zaera, F. & Somorjai, G. A. J. Am. Chem. Soc. 106, 2288-2293 (1984).
50. Grunes, J., Zhu, J., Anderson, E. A. & Somorjai, G. A. J. Phys. Chem. B 106, 11463-11468 (2002).
51. Schlatter, J. C. & Boudart, M. J. Catal. 24, 482-492 (1972).
52. Cortright, R. D., Goddard, S. A., Rekoske, J. E. & Dumesic, J. A. J. Catal. 127, 342-353 (1991).
53. Rioux, R. M., Song, H., Hoefelmeyer, J. D., Yang, P. & Somorjai, G. A. J. Phys. Chem. B 109, 2192-2202 (2005).
54. Somorjai, G. A. & Rioux, R. M. Catal. Today 100, 201-215 (2005).

As used herein and in the appended claims, the singular forms “a”, “and”, and “the” include plural referents unless the context clearly dictates otherwise.

The foregoing detailed description of the present invention is provided for the purposes of illustration and is not intended to be exhaustive or to limit the invention to the embodiments disclosed. Accordingly, the scope of the present invention is defined by the appended claims.

What is claimed is:

1. A core-shell nanoparticle comprising:
a metal-oxide shell comprising an outer surface, an inner surface, and pores, the pores extending from the outer surface to the inner surface, the inner surface forming a void within the metal-oxide shell; and
a nanoparticle filling the void within the metal-oxide shell, the pores allowing gas to transfer from outside the metal-oxide shell to a surface of the nanoparticle.

2. The core-shell nanoparticle of claim 1 wherein the nanoparticle comprises a metal.

3. The core-shell nanoparticle of claim 2 wherein the metal comprises Pt.

4. The core-shell nanoparticle of claim 2 wherein the metal is selected from the group consisting of Pt, Pd, Ru, Rh, Ir, Os, Au, Ag, Cu, Ni, Co, Fe, and a combination thereof.

5. The core-shell nanoparticle of claim 1 wherein the metal-oxide shell comprises SiO₂.

6. The core-shell nanoparticle of claim 1 wherein the metal-oxide shell comprises a first metal oxide selected from the group consisting of SiO₂, Al₂O₃, TiO₂, ZrO₂, Ta₂O₅, and Nb₂O₅, and a combination thereof,

7. The core-shell nanoparticle of claim 1 wherein the nanoparticle is selected from the group consisting of a quantum dot, a cubic nanoparticle, a cuboctahedron nanoparticle, a spherical nanoparticle, a pseudo-spherical nanoparticle, a faceted nanoparticle, a nanorod, a nanowire, a tetrapod, and a branched nanoparticle.

8. The core-shell nanoparticle of claim 1 wherein a majority of the pores have a cross-sectional size within a range from sub-1 nm to 4 nm.

9. The core-shell nanoparticle of claim 1 wherein the metal-oxide shell comprises a mesoporous shell.

10. A method of making a core-shell nanoparticle comprising:
forming a metal-oxide shell on a colloidal nanoparticle that comprises a nanoparticle and a capping agent on the surface of the nanoparticle, thereby producing a precursor core-shell nanoparticle; and
removing the capping agent from the precursor core-shell nanoparticle, thereby producing a core-shell nanoparticle that comprises:
the metal-oxide shell comprising an outer surface, an inner surface, and pores, the pores extending from the outer surface to the inner surface, the inner surface forming a void within the metal-oxide shell; and
the nanoparticle filling the void within the metal-oxide shell, the pores allowing gas to transfer from outside the metal-oxide shell to a surface of the metal nanoparticle.

11. The method of claim 10 wherein the metal-oxide shell comprises SiO₂.

12. The method of claim 10 wherein the metal nanoparticle comprises Pt.

13. The method of claim 10 wherein the capping agent comprises TTAB.

14. The method of claim 10 wherein forming the metal-oxide shell comprises a polymerization process.

15. The method of claim 10 wherein removing the capping agent comprises a calcination process.

16. The method of claim 15 wherein the calcination process comprises heating the nanoparticle to at least 300° C. in an O₂ containing environment.

17. The method of claim 16 wherein the O₂ containing environment comprises air.

18. The method of claim 10 wherein the metal nanoparticle comprises a first metal selected from the group consisting of Pt, Pd, Ru, Rh, Ir, Os, Au, Ag, Cu, Ni, Co, Fe, and a combination thereof.

19. The method of claim 10 wherein the capping agent comprises a first capping agent selected from the group consisting of TTAB, CTAB, alkyl ammonium halide, alkyl amine, alkyl thiol, alkyl phosphine, and PVP.

20. A method of using a nanocatalyst comprising:
providing the nanocatalyst that comprises:
a metal-oxide shell comprising an outer surface, an inner surface, and pores, the pores extending from the outer surface to the inner surface, the inner surface forming a void within the metal-oxide shell; and
a nanoparticle filling the void within the metal-oxide shell, the pores allowing gas to transfer from outside the metal-oxide shell to a surface of the metal nanoparticle; and
introducing reactants in a vicinity of the nanocatalyst, thereby producing a reaction of the reactants that is facilitated or enhanced by the nanocatalyst.

21. The method of claim 20 wherein the reactants comprise CO and O₂.

22. The method of claim 20 wherein the reactants comprise ethylene and H₂.

23. The method of claim 20 wherein the reaction is selected from the group consisting of oxidation, partial oxidation, hydrocarbon cracking, combustion, and hydrogenation.
